(19) United States
(12) Patent Application Publication
(10) Pub. No.: US 2002/0015743 A1
(43) Pub. Date: Feb. 7, 2002

(54) USE OF THE RB1, GINSENOSIDE FOR STIMULATING ELASTIN SYNTHESIS

(76) Inventors: ALAIN MEYBECK, COURBEVOIE (FR); MARC DUMAS, ORLEANS (FR); CATHERINE CHAUDAGNE, VITRY-AUX-LOGES (FR)

Correspondence Address: DENNISON MESEROLE SCHEINER & SCHULTZ, 1745 JEFFERSON DAVIS HIGHWAY, SUITE 612, ARLINGTON, VA, 22202-3417, US

(21) Appl. No.: 09/463,412

(22) PCT Filed: Aug. 7, 1998
(86) PCT No.: PCT/FR98/01770

(30) Foreign Application Priority Data
Aug. 8, 1997 (FR) 97/10204

(51) Int. Cl. A61K 35/78; A61K 31/56

(52) U.S. Cl. 424/728; 514/182

(57) ABSTRACT

The invention concerns novel uses in cosmetics or pharmaceutics, in particular in dermatology, of Rb1 ginsenoside(G-Rb1) and plant extracts containing same, to stimulate elastin synthesis by dermal fibroblasts. The invention also concerns cosmetic or pharmaceutical, in particular dermatological, compositions containing said ginsenoside or plant extracts for stimulating the elastin synthesis by dermal fibroblasts. Said compositions advantageously contain an extract of Asiatic ginseng or of American ginseng.

The invention relates to novel uses of ginsenoside Rb1 as agent intended to stimulate the synthesis of elastin.

This invention finds applications essentially in the field of cosmetics and pharmacy, notably in the field of dermatology.

Generally, when it is sought to fight effectively against skin ageing, it is attempted to treat the skin with products having an anti-radical activity which are susceptible in particular to trapping the free radicals which are responsible, in part, for the harmful effects leading to an ageing of the skin.

It is known that in the dermis, the elastic fibres form a network which contributes to the elasticity of the skin, and, consequently, to its tonicity and to its firmness. Elastin is the major component of these fibres. Elastin is initially synthesised by the fibroblasts in the form of a soluble polypeptide of 70 kDa, tropoelastin. The formation of the microfibrils then takes place by intramolecular bonds called desmosins between the lysine residues and the polypeptide chains. These bonds confer to the elastin a great insolubility.

Furthermore, it is known that the content of elastin drops in the human organism after the age of 25 years. There are good reasons to believe that the appearance of wrinkles could be, inter alia, linked to a progressive disappearance of dermal elastin. This is the reason why certain cosmetologists have proposed treating the skin with preparations of products derived from elastin. It has thus been proposed to use compositions containing hydrolysed elastin in order to render it soluble. However, the drawback of such products is linked to the too high molecular weight of the hydrolysed elastin which penetrates with difficulty across the skin.

Another much more interesting solution, and which constitutes a real progress, is to promote the synthesis of elastin directly by the cells of the skin. Two types of products are hitherto known for having such an effect. It is, on the one hand, a matter of ethocyne the activity of which has been described by L. C. FORD et al. (IFSCC Conference, Barcelona, 1986, Vol. 1, p. 987-989) and on the other hand, ascorbic acid and its derivatives the activity of which is described in the International Application WO 96/19099.

The Panax family comprises numerous species and varieties, the most known of which are:

Panax ginseng (C. A. Meyer) which is the most frequently used for its medical virtues,

Panax notoginseng,

Panax pseudo-ginseng (subsp. himalaicus),

Panax japonicus (var. major; var. angustifolius),

Panax quinquefolium,

Panax trifolius,

Panax zingiberensis,

Panax stipuleanatus.

The Panaxes essentially originate from three countries

Japan

China

Korea.

The species and varieties cultivated in these countries are different and experience various geo-climatic conditions.

Very different saponin contents are noticed in the various parts of the plant. The root part is the most frequently used and is generally reputed to be the most active.

The highest saponin content is observed in the extremity of the root (ninjin in Japanese) and in the rootlets (hairy roots ; keninjin, in Japanese).

The best known Panax is Panax ginseng or ginseng.

It has a very particular composition of saponins (called ginsenosides).

Each species or variety of Panax has a particular composition of ginsenosides, which confers to the extracts which originate from it properties which are different from those of the extracts of other Panaxes.

Panax notoginseng (Sanchi in Chinese) and Panax quinquefolium (American ginseng) differ greatly from Panax ginseng. They are particularly rich in ginseno side Rb1, designated by G-Rb1 of formula:

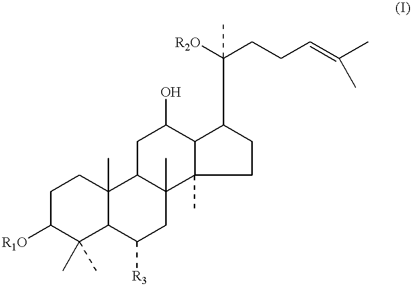

in which

R1=Glc(2-1)Glc,

R2=Glc(6-1)Glc,

R3=H

wherein Glc designates a β-D glucopyranosyl group.

An activity of this ginsenoside Rb1 has been described in the International Application PCT WO 94/06402 and, more particularly, extracts of Panax notoginseng or San-chi particularly rich in this ginsenoside for the care of the hair, particularly for preventing the fall thereof.

Further, the inhibitory action is known of this same ginsenoside on the proliferation of the keratinocytes. (Nack-in-Kim et al. J.Invest.Dermatol., 101 (3), 491, 1993.)

This same ginsenoside Rb1 is, furthermore, particularly abundant in extracts of Panax quinquefolium.

Panax quinquefolium is above all used as a tonic (see article referenced Hort. Technology 5 (1), 27-34, 1995).

Another ginsenoside, ginsenoside Rb2, has been found to be effective to stimulate the synthesis of glycosaminoglycans (GAG) by the fibrocytes (Tanaka H. et al., Nippon Koshohin Kagakkaishi, 15 (3), 132-5, 1991 and Tanaka H. et al. Fragrance J., 19 (8), 90-2, 1991).

Furthermore, studies have shown that ginsenosides of ginseng have a positive effect upon the elasticity of the skin of post-menopause women (A. Gezzi et al. Fitoterapia, 57 (1), 15-28, 1986. S. B. Curri et al. Fitoterapia, 57, 4, 217-222, 1986). In parallel, these authors have shown a positive effect of an extract of ginseng having 14% of ginsenosides upon the hydration of the horny layer of the skin of women. In the case of the extract of ginseng used which contains other ginsenosides as major saponins and in which the ginsenoside Rb1 is in low concentration, the main effect seems to be an effect of hydration which manifests itself by a better elasticity.

Furthermore, Russian patent SU 839542 describes a cosmetic cream which contains, inter alia, an extract of ginseng. This cream is intended to prevent the withering of the skin. This cream improves the blood circulation, the metabolism of proteins and fats, the morphological structure of the skin and its elasticity.

The inventors of the present application have now demonstrated the specific effect of ginsenoside Rb1, named G-Rb1, to stimulate the biosynthesis of elastin by the fibroblasts of the dermis, particularly the fibroblasts of the human dermis.

Thus, the aim of the present invention is to solve the novel technical problem consisting of providing a solution which enables improving the skin elasticity by promoting the synthesis of elastin, according to a solution of simple conception, which can be used easily on an industrial scale, while at the same time enables a cosmetic or pharmaceutical use, notably a dermatological use.

According to one of its essential characteristics, the invention relates to the cosmetic use of ginsenoside Rb1 (hereinafter also designated as G-Rb1) or of plant extracts containing same as active agent of a composition intended to stimulate the synthesis of elastin by the fibroblasts of the dermis.

According to another essential characteristic, the invention relates to the use of the same ginsenoside Rb1 or of plant extracts containing same for the preparation of a pharmaceutical composition, notably a dermatological composition, intended for the treatment of disorders linked to an insufficiency in the synthesis of elastin by the fibroblasts of the dermis.

In these two types of use, the result of the stimulation of the synthesis of elastin is to improve the skin elasticity and tonicity or to promote the firming of the skin.

According to a particularly advantageous variant of the invention, the cosmetic as well as pharmaceutical compositions, notably dermatological compositions, of the invention will be able to comprise, in addition to the ginsenoside Rb1, at least one other saponin of the ginsenoside type.

Thus, according to a particularly advantageous variant of the invention, it will be possible for ginsenoside Rb1 to be introduced in the composition in the form of an extract of a plant of the Panax type.

Extracts containing:

2 to 60% by weight of saponin G-Rb1,

2 to 60% by weight of saponin G-Rg1,

0 to 15% by weight of saponin G-Rd,

0 to 15% by weight of saponin N-R1,

1 to 10% by weight of saponin G-Re,

will be cited as examples of extracts which are particularly useful for the implementation of the invention.

More preferably, an extract containing:

10 to 60% by weight of saponin G-Rb1,

10 to 60% by weight of saponin G-Rg1,

0 to 15% by weight of saponin G-Rd,

0 to 15% by weight of saponin N-R1,

1 to 10% by weight of saponin G-Re,

with respect to the total weight of said extract, will be selected.

According to yet another variant, an extract will be selected in which ginsenoside G-Rb1 is the major saponin constituent.

As seen above, plants of the Panax notoginseng and Panax quinquefolium type are two plants of the Panax type which are particularly rich in ginsenoside Rb1. It is therefore to these particular plants to which recourse will preferably be made for the preparation of cosmetic or pharmaceutical compositions, notably dermatological compositions, of the invention.

For these two types of plants, their subterranean parts will preferably be used.

The extract is advantageously obtained by extraction of the plant matter with a polar solvent or a mixture of polar solvents.

In this context, it will be possible for the classical extraction procedures well known to the person skilled in the art to be used. Particularly, the extraction can be carried out on the ground plant matter.

Advantageously, the extraction is carried out on the rhizomes.

The extraction can also be carried out on the plant tissues in culture (in vitro culture of roots or calluses).

The ground plant matter is introduced in the extraction solvent, preferably constituted by the solvent or the mixture of polar solvents mentioned above. The extraction can be renewed several times until the matter has run out, in accordance with the procedures well known to the person skilled in the art. The extraction can be carried out at ambient temperature, or in the hot, and notably at the reflux of the solvent. The proportion by weight between the matter to be extracted and the solvent can vary within large limits. More specifically, it can be between 1:5 and 1:100, and, preferably, between 1:10 and 1:20.

According to a particular embodiment of the invention, the plant extract containing the above-mentioned saponins is obtained according to the method described below as an indication but not in a limiting way. The dry matter is extracted by means of a solvent selected from the group constituted by : water, alcohols comprising preferably 1 to 4 carbon atoms, and mixed solvents based on any mixture of the solvents mentioned above.

Preferably, an alcohol such as methanol, ethanol, propanol, isopropanol, propyleneglycol or butyleneglycol, will be used or a mixture of these alcohols, or even a hydro-alcoholic mixture.

A solvent selected from the group constituted by methanol, ethanol, 1,3-butyleneglycol and mixtures of these solvents by themselves or with water will advantageously be selected as solvent for carrying out this extraction.

Preferably, a first extraction, known as primary extraction, is carried out at reflux under atmospheric pressure for a period of about 30 minutes. At the end of the extraction, the phase of the solvent containing the extract is left to cool and then it is filtered. Advantageously, the residue is extracted once or twice again according to the same protocol, the filtrates are combined and then concentrated and/or evaporated to dryness under reduced pressure. A first extract according to the invention is thus obtained which is rich in saponins. This extract can also be lyophilised.

According to a particular variant, a mixture of saponins according to the invention is prepared from a first concentrated or dry extract mentioned above in operating as indicated below. The first extract mentioned above, which was obtained preferably with a primary solvent easily removable by evaporation, such as methanol or ethanol or even a methanol-water or ethanol-water mixture, is introduced and then agitated in an a polar solvent preferably miscible with the primary extraction solvent, such as an ether or a ketone of low molecular weight, in particular ethyl ether or isopropyl ether, acetone or methyl ethyl ketone. The quantity of apolar solvent is, by weight, generally 5 to 100 parts per part of primary extract. The insoluble and/or the precipitate formed contains mainly a mixture of saponins usable according to the invention.

If desired, the mixture of saponins obtained above can be purified once again by any procedure at the reach of the person skilled in the art.

In particular, the insoluble and/or the precipitate mentioned above is put back into solution in about 20 times its weight of water. The aqueous solution is then extracted 3 to 4 times by an alcohol not very soluble in water, such as butanol saturated in water, for example in a 1/1 proportion by volume at each extraction operation. The alcoholic phases are combined and evaporated under reduced pressure. The residue is dissolved in about 10 times its weight of water, the solution is then dialysed against pure water for 4 to 5 days. The content of the dialysis cell is lyophilised. Optionally, in order to improve the purification of the mixture of saponins obtained, the lyophilisate is dissolved in methanol, and then cast into ethyl ether. The precipitate formed is collected.

According to another advantageous embodiment, the ginsenoside Rb1 or the extracts mentioned above are used preferably via the topical route at a concentration between 0.001% and 5% with respect to the total weight of a composition containing same in an appropriate excipient, vehicle or support, preferably cosmetically or pharmaceutically, notably dermatologically acceptable. A preferred concentration is between 0.01 and 1% by weight with respect to the total weight of the composition containing same.

According to an advantageous embodiment of the invention, the cosmetic or pharmaceutical composition, notably dermatological composition, of the invention contains, further, ascorbic acid, particularly L-ascorbic acid or vitamin C or its isomer, erythorbic acid or one of their salts or esters, designated under the generic name of ascorbic derivatives.

According to an advantageous embodiment of the invention, the ginsenoside Rb1 or the extracts according to the invention are used in combination with an effective amount of an active principle stimulating the synthesis of the components of the extracellular matrix of the dermis, amongst which collagen, particularly collagen I and collagen III, and glycosaminoglycans, particularly hyaluronic acid will be cited.

Advantageously, an extract of Centella asiatica, or an extract of ginseng, particularly an extract containing ginsenoside Ro, will be used as active principle stimulating the synthesis of collagen, particularly of collagen I and of collagen III, madecassoside, said active principle being at a concentration between 0.01% and 5% by weight with respect to the total weight of the final composition.

A plant extract such as an extract of Filicium decipiens, particularly an extract of the bark of the root of this plant, described in the French patent application 95 07708, or an extract of Eryobotria japonica described in the French patent application 96.00018, or a growth factor, particularly PDGF (platelet derived growth factor), growth factor derived from blood platelets, will be used as examples of an active principle stimulating the synthesis of glycosaminoglycans.

According to another advantageous embodiment, ginsenoside Rb1 or the above-mentioned extracts are used in combination with a vitamin, particularly vitamin A, its palmitic, propionic or acetic ester or vitamin E and its derivatives, particularly at a concentration between 0.0001 and 5% by weight with respect to the total weight of the composition.

According to a third aspect, the present invention also covers a method of cosmetic or therapeutic treatment of the skin which is intended to prevent or to correct a loss of skin elasticity, tonicity or firmness, characterised in that it comprises the application on the areas of the skin to be treated an effective amount of ginsenoside Rb1 or of an extract containing same, to stimulate la synthesis of elastin by the fibroblasts, said product being incorporated in a cosmetically or pharmaceutically acceptable excipient.

For the implementation of such a method, an extract of the subterranean parts of Panax notoginseng or Panax quinquefolium will advantageously be selected.

The variants of this method also result clearly from the variants of the use mentioned above.

It results from the foregoing that ginsenoside Rb1 or the extract containing same are precious for the preparation of a cosmetic or pharmaceutical composition, notably dermatological composition, wherein an improvement of the skin elasticity is sought by stimulation of the synthesis of elastin.

The person skilled in the art thus understands the major interest of the invention which promotes the synthesis of elastin and its ease of use by virtue of a topical application on the skin.

Other aims, characteristics and advantages of the invention will appear clearly in the light of the explanatory description which will follow, made with reference to the Examples given hereafter simply as an illustration and which in no way limit the scope of the invention.

In the Examples, all the percentages are given by weight unless otherwise indicated.

### EXAMPLES

### Example 1

Preparation of an Extract of Panax notoginseng

200 g of dried roots of Panax notoginseng (San-Chi) are ground, and then extracted with 2 liters of methanol under reflux for 30 minutes. The methanolic phase is left to cool to ambient temperature, and is then filtered. The solid residue is extracted once again, in the same manner, with 2 liters of methanol.

The two methanolic filtrates are combined, and the solvent is evaporated off under vacuum until a solid residue is obtained.

This solid is then treated with 100 ml of acetone. The insoluble part in acetone is recovered and placed in solution in 50 ml of methanol. Ethyl ether is then added to this solution until a precipitate is obtained.

This precipitate is filtered off. It constitutes a mixture of saponins of Panax notoginseng according to the invention, containing in particular ginsenoside G-Rb1. This mixture, designated as extract E1, will be used in the tests reported in Example 3 below (see Table 2).

### Example 2

Preparation of Extracts of Panax quinquefolium

a) Preparation of a Methanolic Extract

200 g of dried roots of Panax quinquefolium (American Ginseng) are ground, and then extracted with 2 liters of methanol under reflux for 30 minutes.

As in the preceding Example, the methanolic phase is left to cool, then the extraction operation is started again twice.

The various filtrates are then combined and are evaporated to dryness under vacuum in a rotary flask.

A methanolic extract of Panax quinquefolium is thus obtained which is rich in saponins, and which contains in particular G-Rb1. This extract, named extract E2, will be used in the tests reported in Example 3 below (see Table 3).

b) Preparation of a Mixture of Saponins extracted from Panax quinquefolium

The extract E2 obtained above is treated with 100 ml of acetone. The insoluble part is filtered of and dissolved in 50 ml of methanol. Ethyl ether is added to the solution obtained until a precipitate is formed which is filtered and dried.

The solid residue (named E3) obtained is constituted essentially of a mixture of saponins of Panax quinquefolium, which contains in particular ginsenoside G-Rb1. This extract is itself as well used in the tests reported in Example 3 (see Table 3).

c) Preparation of a Fraction of Extract of Panax quinquefolium, Rich in Ginsenoside G-Rb1

The mixture of saponins obtained in the preceding step is dissolved in 10 ml of water. This solution is adsorbed onto 10 g of Celite which is left to dry under a hood. When the powder is dry, it is placed on top of a silica column. A chromatography is then carried out following usual techniques well known to the person skilled in the art. The saponins are then successively eluted with a chloroform-methanol-water mixture 50/50/3. Various fractions are collected, and the fraction richest in ginsenoside G-Rb1 is selected by carrying out thin layer chromatographies in comparison with a commercial control.

This fraction, named E4, rich in ginsenoside G-Rb1, is used in the tests reported in Example 3 below (see Table 3).

### Example 3

Example of test showing the activity of ginsenoside G-Rb1 and of extracts containing same on the stimulation of the synthesis of elastin by human skin fibroblasts.

Material and Methods

Origin of the Cells:

A strain of normal human fibroblasts (NHF) is used in this study which originates from surgical skin of the face of a 50 year old woman (NHF-50 years) and from a 47 year old woman (NHF-47 years). The cells had been obtained by the method of explants as described by M. Dumas et al. in the article entitled “in vitro biosynthesis of type I and III collagens by human dermal fibroblasts from donors of increasing ages” in Mech. Ageing Dev. 73 (1994), pages 179-187.

Use of the Cell Cultures:

These normal human skin fibroblasts thus obtained are sown in the wells of a multi-well culture box, in an E 199 culture medium, in which 2 millimoles per liter of L-glutamine have been added.

Further, for a certain number of tests, the culture medium contains sodium ascorbate at concentrations of 15 or 25 micromoles per liter.

24 hours after sowing, certain wells receive ginsenoside Rb1 or an extract of Panax 1 to 10 μg/ml via a solution in DMSO so that the final DMSO concentration be 0.1%.

Six wells are used each time per product and per concentration, as well as six wells do not receive any product to be tested for the control cultures. These control cultures only receive DMSO at a final concentration of 0.1% by weight.

The culture is continued for 48 h.

After 48 h of culture as indicated above, the elastin secreted is determined on the supernatants of the culture, by an ELISA method derived from that used for the determination of collagen, as described by M. Dumas et al in the publication cited above, except that a commercial human anti-elastin polyclonal antibody was used instead of the human anti-collagen I and III polyclonal antibodies.

This determination is naturally also carried out on the control cultures not receiving any product to be tested.

A standard range made from commercial soluble elastin enables converting into nanograms of elastin the values of optical density (OD) obtained from the culture supernatants.

Determination of Proteins:

In parallel with the determination of the elastin, a determination of the cell proteins is carried out on the cultures of fibroblasts, after removal of the supernatant, in order to bring the amount of elastin determined to a fixed amount of cell proteins. The determination is carried out by using a BCA-1 kit (Bicinchoninic acid kit, marketed by Sigma, France) after dissolution of the cell plug with 0.1 N sodium hydroxide solution.

Expression of the Results and Statistics:

The results are expressed in nanograms of elastin secreted per microgram of cellular proteins. The activity A of stimulation, expressed in percentage, is calculated according to the following formula:

$$A = \frac{q - qo}{q0} \times 100$$

in which:

q represents the amount of elastin secreted by the treated NHFs,

qo represents the amount of elastin secreted by the control NHFs.

The results obtained on the treated cultures (n=6) and controls (n=6) are compared with the aid of the Student t test for non-paired series. Any value of p<0.05 will indicate that a significant difference exists between the secretion of elastin of the control NHFs and those treated with the products of the invention.

Tables 1A, 1B, 2 and 3 below report the results obtained with ginsenoside Rb1 and the various extracts E1, E2, E3 and E4 obtained according to Examples 1 and 2.

More Specifically:

Table 1A gives the results obtained with a commercial ginsenoside Rb1 introduced in the cultures of fibroblasts of the face of a woman of 47 years (NHF-47 years),

Table 1B gives the results obtained with the same commercial ginsenoside introduced in the cultures of fibroblasts of the face of a woman of 50 years (NHF-50 years),

Table 2 gives the results obtained with the extracts of saponins of Panax notoginseng obtained according to Example 1 (E1), introduced in the cultures of fibroblasts of the face of a woman of 50 years (NHF-50 years),

Table 3 gives the results obtained with the extracts of Panax quinquefolium (noted E2, E3 and E4) obtained according to Example 2.

TABLE 1A
ACTIVITY of GINSENOSIDE Rb1 ON THE SYNTHESIS
of ELASTIN (NHF-47 years)

| | ng Elastin/ | Activity in | Student t test |
|---|---|---|---|
| Experimental conditions | μg prot/48 h | % | values of p |
| C. + DMSO 0.1% | 19.7 + 1.6 | 0 | |
| G-Rb1 10 μg/ml | 25.8 + 2.2 | 31 | 0.00033 |
| C. + DMSO 0.1% + | 33.9 + 1.9 | 0 | |
| Asc. 15 μM | | | |
| G-Rb1 10 μg/ml + | 39.3 + 2.8 | 16 | 0.0039 |
| Asc. 15 μM | | | |
| C. + DMSO 0.1% + | 35.1 + 2.9 | 0 | |
| Asc. 25 μM | | | |
| G-Rb1 10 μg/ml + | 38.8 + 6.8 | 10 | 0.2663 |
| Asc. 25 μM | | | |

C. = Control; Asc. = Ascorbate

TABLE 1B
ACTIVITY of GINSENOSIDE Rb1 ON THE SYNTHESIS
OF ELASTIN (NHF-50 years)

| | ng Elastin/ | Activity in | Student t test |
|---|---|---|---|
| Experimental conditions | μg prot/48 h | % | values of p |
| C. + DMSO 0.1% + | 23.9 + 2.4 | 0 | |
| Asc. 25 μM | | | |
| G-Rb1 10 μg/ml + | 28 + 2.2 | 17 | 0.0125 |
| Asc. 25 μM | | | |
| C. + DMSO 0.1% + | 21.7 + 1.2 | 0 | |
| Asc. 25 μM | | | |
| G-Rb1 10 μg/ml + | 26.3 + 3.7 | 21 | 0.0154 |
| Asc. 25 μM | | | |
| C. + DMSO 0.1% + | 27.3 + 2.6 | 0 | |
| Asc. 25 μM | | | |
| G-Rb1 10 μg/ml + | 32 + 4.3 | 17 | 0.0449 |
| Asc. 25 μM | | | |

C. = Control; Asc. = Ascorbate

TABLE 2
ACTIVITY OF AN EXTRACT OF AN EXTRACT OF PANAX
NOTOGINSENG ON THE SYNTHESIS OF ELASTIN
(NHF-50 years)

| | ng Elastin/ | Activity in | Student t test |
|---|---|---|---|
| Experimental conditions | μg prot/48 h | % | values of p |
| C. + DMSO 0.1% + | 33.4 + 4.3 | 0 | |
| Asc. 25 μM | | | |
| E1 1 μg/ml + | 38.8 + 3.4 | 16 | 0.038 |
| Asc. 25 μM | | | |
| E1 2.5 μg/ml + | 39.7 + 2.9 | 19 | 0.015 |
| Asc. 25 μM | | | |
| C. + DMSO 0.1% + | 32.2 + 7.4 | 0 | |
| Asc. 25 μM | | | |
| E1 1 μg/ml + | 35.6 + 5.9 | 11 | 0.404 |
| Asc. 25 μM | | | |
| E1 2.5 μg/ml + | 40.9 + 4.7 | 27 | 0.0362 |
| Asc. 25 μM | | | |

C. = Control
Asc. = Ascorbate

TABLE 3
ACTIVITY of EXTRACTS of PANAX QUINQUEFOLIUM ON THE
SYNTHESIS OF ELASTIN
(NHF-50 years)

| | ng Elastin/ | Activity in | Student t test |
|---|---|---|---|
| Experimental conditions | μg prot/48 h | % | values of p |
| C. + DMSO 0.1% + | 21.7 + 1.2 | 0 | |
| Asc. 25 μM | | | |
| E2 1 μg/ml + | 22.9 + 2.5 | 5.5 | 0.33 |
| Asc. 25 μM | | | |
| E2 2.5 μg/ml + | 25.2 + 3.5 | 16 | 0.044 |
| Asc 25 μM | | | |
| C. + DMSO 0.1% + | 23.6 + 1.7 | 0 | |
| Asc. 25 μM | | | |
| E3 1 μg/ml + | 24.3 + 1.7 | 3 | 0.473 |
| Asc. 25 μM | | | |
| E3 2.5 μg/ml + | 29.4 + 4.2 | 25 | 0.01 |
| Asc. 25 μM | | | |
| Asc. 25 μM + E4 1 μg/ml | 27.9 + 1.1 | 18 | 0.0004 |
| C. + DMSO 0.1% + | 33.4 + 4.3 | 0 | |
| Asc. 25 μM | | | |
| E2 1 μg/ml + | 35.3 + 6 | 5.7 | 0.546 |
| Asc. 25 μM | | | |
| E2 2.5 μg/ml + | 41.9 + 3.8 | 25 | 0.005 |
| Asc. 25 μM | | | |
| C. + DMSO 0.1% + | 27.3 + 2.6 | 0 | |
| Asc. 25 μM | | | |
| E3 1 μg/ml + | 29.7 + 4.1 | 8.8 | 0.239 |
| Asc. 25 μM | | | |
| E3 2.5 μg/ml + | 31.4 + 2.9 | 15 | 0.025 |
| Asc. 25 μM | | | |
| Asc. 25 μM + E4 1 μg/ml | 30.7 + 3.7 | 12 | 0.094 |

C. = Control; Asc. = Ascorbate

Conclusion

As it emerges from Tables 1 to 3, ginsenoside Rb1, the extracts of Panax notoginseng as well as the extracts of Panax quinquefolium strongly stimulate the synthesis of elastin by the fibroblasts. This stimulation augments with the concentration of the product tested.

It is thus demonstrated that the use of ginsenoside Rb1, of an extract of Panax notoginseng or of an extract of Panax quinquefolium, enables stimulating the synthesis of elastin by the fibroblasts in a significant and particularly unexpected manner.

Examples of applications.

The compositions are expressed below in percentages by weight.

### Example 4

Gel for Face Care:

ginsenoside Rb1 0.2

hydrogenated lecithin 1

hyaluronic acid 1

Carbopol 941® 1.25

water+preservative+triethanolamine qsp 100.

By local application twice a day on the base of the face and the neck, this care product improves and maintains the elasticity of the skin of the face and reduces thereon the wrinkles and small wrinkles.

### Example 5

Firming Cream for the Face:

propyleneglycol 1

glycerylstearate PEG 100 2

glycerylstearate sodium lauryl sulphate 4

glyceryl stearate 3

cetyl alcohol 1

caprilic/capric triglyceride 3

octyl stearate 2

avocado oil 1

camellia oil 1

ascorbyl palmitate 5

vitamin A palmitate 0.01

wheat glycoceramides 0.1

lactic acid 0.5

Carbopol 940® 0.5

saponins of Panax notoginseng (E1) 0.5

water+preservative+perfumes qsp 100.

This cream is applied morning and night and gives back the elasticity to the skin of the face which thus gives a better appearance.

### Example 6

Body Care Milk:

vegetable oil 2

caprylic/capric triglyceride 5

vitamin E acid 0.56

carbomer 0.5

xanthan gum 0.20

hyaluronic acid 0.15

extract of Panax quinquefolium (E2) 0.5

extract of centella asiatica 0.1

extract of green tea 0.05

water+preservative+refreshing agent+perfume qsp 100

By application on the bust in the evening in a cure of 20 days, with massage in order to enable the tissues of the bust to conserve their elasticity.

### Example 7

Lotion for Improving the Elasticity of the Skin of the Face.

Saponins of Panax quinquefolium (E3) 0.3

1% hyaluronic acid aqueous solution 1

glycerol 2

water+preservative qsp 100.

This lotion, for improving the elasticity of the skin, can be used after make-up removal for women or after shaving for men.

The invention also covers every technical equivalent of the means described, as well as the various combinations thereof.

1. Cosmetic use of ginsenoside Rb1 (G-Rb1) or of plant extracts containing same as active agent of a composition intended to stimulate the synthesis of elastin by the fibroblasts of the dermis.

2. Use of ginsenoside Rb1 (G-Rb1) or of plant extracts containing same for the preparation of a pharmaceutical composition, notably a dermatological composition, intended for the treatment of disorders linked to an insufficiency in the synthesis of elastin by the fibroblasts of the dermis.

3. Use according to one of claims 1 or 2, characterised in that said composition contains 0.001 to 5% by weight of said ginsenoside or of said extract.

4. Use according to one of claims 1 to 3, characterised in that said composition contains, in addition to the ginsenoside Rb1, at least one other saponin of the ginsenoside type.

5. Use according to one of claims 1 to 4, characterised in that said ginsenoside G-Rb1 is introduced into said composition in the form of an extract of a plant of the Panax type.

6. Use according to claim 5, characterised in that said extract contains
2 to 60% by weight of saponin G-Rb1,
2 to 60% by weight of saponin G-Rg1,
0 to 15% by weight of saponin G-Rd,
0 to 15% by weight of saponin N-R1,
1 to 10% by weight of saponin G-Re,
with respect to the total weight of said extract.

7. Use according to one of claims 5 or 6, characterised in that said extract contains:
10 to 60% by weight of saponin G-Rb1,
10 to 60% by weight of saponin G-Rg1,
0 to 15% by weight of saponin G-Rd,
0 to 15% by weight of saponin N-R1,
1 to 10% by weight of saponin G-Re,
with respect to the total weight of the extract.

8. Use according to one of claims 5 to 7, characterised in that said ginsenoside G-Rb1 is the saponin mainly contained in said extract.

9. Use according to one of claims 5 to 8, characterised in that said plant of the Panax type is Panax notoginseng or Panax quinquefolium.

10. Use according to one of claims 5 to 9, characterised in that said extract is an extract of Panax notoginseng, preferably of subterranean parts of the plant.

11. Use according to one of claims 5 to 9, characterised in that said extract is an extract of Panax quinquefolium, preferably an extract of a subterranean part of this plant.

12. Use according to one of claims 5 to 11, characterised in that said extract is obtained by extraction by means of a polar solvent or of a mixture of polar solvents.

13. Use according to one of claims 5 to 12, characterised in that said extract is obtained by extraction by means of a solvent or of a mixture of solvents selected from the group constituted by water, alcohols comprising preferably 1 to 4 carbon atoms, particularly methanol, ethanol, propanol, isopropanol, propyleneglycol and butyleneglycol.

14. Use according to claim 13, characterised in that the solvent or mixture of solvents is selected from the group constituted by methanol, ethanol, 1,3-butyleneglycol and mixtures of these solvents by themselves or with water.

15. Use according to one of claims 1 to 14, characterised in that said composition contains, further, ascorbic acid, particularly L-ascorbic acid or vitamin C or its isomer erythorbic acid or one of their salts or esters, designated under the generic name of ascorbic derivatives.

16. Use according to one of claims 1 to 14, characterised in that said composition contains, further, an effective amount of an active principle stimulating the synthesis of the components of the extracellular matrix of the dermis, particularly of collagen, particularly of collagen I and of collagen III, and of glycosaminoglycans.

17. Use according to claim 16, characterised in that said composition contains an active principle stimulating the synthesis of collagen, particularly of collagen I and of collagen III, selected from the group constituted by madecassoside, an extract of centella asiatica and an extract of ginseng, particularly ginsenoside Ro, at a concentration by weight of 0.01% to 5%.

18. Use according to claim 16, characterised in that the composition contains an active principle stimulating the synthesis of glycosaminoglycans, selected from the group constituted by an extract of Filicium decipiens, particularly an extract of the bark of the root of this plant, an extract of Eriobotrya japonica and PDGF.

19. Use according to one of claims 1 to 18, characterised in that said composition contains, further, vitamins, particularly vitamin A, its palmitic, propionic or acetic ester or vitamin E and its derivatives, at a concentration by weight of 0.0001 to 5% with respect to the total weight of said composition.

20. Method of cosmetic treatment intended to prevent or to correct a loss of skin elasticity, tonicity or firmness, characterised in that it comprises the application on the areas of the skin to be treated of an effective amount of ginsenoside Rb1 or of a plant extract containing same to stimulate the synthesis of elastin by the fibroblasts of the dermis, said ginsenoside being incorporated in a cosmetically acceptable excipient.
